(19) United States
(12) Patent Application Publication
(10) Pub. No.: US 2005/0245957 A1
(43) Pub. Date: Nov. 3, 2005

(54) Biasing stretch receptors in stomach wall to treat obesity

(75) Inventors: Warren L. Starkebaum, Plymouth, MN (US); Martin T. Gerber, Maple Grove, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/836,549

(22) Filed: Apr. 30, 2004

(51) Int. Cl. A61M 29/00

(52) U.S. Cl. 606/191

(57) ABSTRACT

Medical devices and methods are designed to bias stretch receptors in the stomach wall of a patient to treat obesity. Biasing of the stretch receptors by pre-stretching induces an early sensation of satiety, causing the patient to consume less food. Biasing of the stretch receptors can be achieved by the placement of bulking devices within the wall of the stomach, e.g., in the mucosa, submucosa or muscle layer. The bulking devices may be expandable and, in some embodiments, may take the form of a hydrogel prosthesis that expands following implantation in a wall of the stomach.

### FIELD OF THE INVENTION

The invention relates to medical devices and methods for treatment of obesity.

### BACKGROUND

Obesity is a major health concern in the United States and other countries. A significant portion of the population is overweight with the number increasing every year. Obesity is one of the leading causes of preventable death. Obesity is associated with several co-morbidities that affect almost every body system. Some of these co-morbidities include: hypertension, heart disease, stroke, high cholesterol, diabetes, coronary disease, breathing disorders, sleep apnea, cancer, gallstones, and musculoskeletal problems. An obese patient is also at increased risk of developing Type II diabetes.

Multiple factors contribute to obesity, including physical inactivity and overeating. A variety of medical approaches have been devised for treatment of obesity. Existing therapies include diet, exercise, appetite suppressive drugs, metabolism enhancing drugs, surgical restriction of the gastric tract, and surgical modification of the gastric tract. In general, surgery is reserved for patients in whom conservative measures, such as monitoring caloric intake or controlling appetite with appetite suppressants, have failed. In addition, surgery is generally reserved for patients who are seriously, and sometimes morbidly, overweight.

There have been many surgical approaches to obesity. For example, some patients have received implantation of one or more bulking prostheses to reduce stomach volume. A bulking prosthesis resides within the stomach and limits the amount of food the stomach can hold, theoretically causing the patient to feel a sensation of satiety. U.S. Published patent application No. 20030040804 to Stack et al., for example, describes a tubular prosthesis that is designed to induce sensations of satiety within a patient.

Another approach is restrictive surgery, which surgically makes the stomach smaller by removing or closing a section of the stomach. This procedure also reduces the amount of food the stomach can hold, causing the patient to feel full. U.S. Published patent application No. 20020183768 to Deem et al., which describes a recent proposal for treating obesity, discloses various techniques for reducing the size of the stomach pouch to limit caloric intake, as well as to provide an earlier feeling of satiety.

Another surgical procedure to treat obesity is the gastric bypass procedure. In the gastric bypass procedure, the surgeon creates a small stomach pouch to restrict food intake and constructs a bypass of the duodenum and other segments of the small intestine. This procedure limits the amount of food that can be ingested and subsequently digested or absorbed.

Surgical procedures for treatment of obesity, such as those described above, tend to be highly invasive, and each form of surgery may involve complications. Restrictive surgery may entail a risk of vomiting, for example, and gastric bypass surgery may result in unpleasant consequences known as “dumping syndrome.”

Another surgical technique is described in U.S. Pat. No. 6,427,089 to Knowlton. In particular, Knowlton describes a surgical technique for causing a contraction or reduction in the volume of the stomach by the delivery of thermal energy to the stomach wall. According to Knowlton, the technique relies on a microwave device to heat a submucosal layer of tissue within the stomach wall without thermal damage of the mucosa of the stomach. A resulting thermal lesion causes contraction of the preexisting collagen matrix of the stomach wall.

A further technique is described in PCT Publication No. WO 00/69376 to Edwards in which nerves responsible for the sensations of hunger are ablated by applying energy to the interior mucosal lining of the stomach. The mucosal lining of the stomach, which is responsible for protecting the stomach tissue and producing stomach acid necessary for digestion, is ablated along with the specified nerves.

U.S. Pat. No. 6,540,789 to Silverman describes a technique for treatment of obesity involving introduction of an implant material into the stomach wall in the vicinity of the pyloric sphincter to inhibit emptying of the stomach. Silverman also describes introduction of an implant material to reduce distensibility and contractility of the stomach.

Table 1 below lists documents that disclose techniques for treatment of obesity.

TABLE 1

| Patent Number | Inventors | Title |
|---|---|---|
| 20020183768 | Deem et al. | Obesity treatment tools and methods |
| 20030040804 | Stack et al. | Satiation devices and methods |
| WO/0187335 | Uhlman et al. | Method for selectively inhibiting |
| | | ghrelin action |
| 6,427,089 | Knowlton | Stomach treatment apparatus |
| | | and method |
| 5,782,798 | Rise | Techniques for treating eating |
| | | disorders by brain stimulation |
| | | and drug infusion |
| WO 00/69376 | Edwards | Surgical weight control device |
| 5,423,872 | Cigaina | Process and device for treating |
| | | obesity and syndromes related to |
| | | motor disorders of the |
| | | stomach of a patient |
| 5,188,104 | Wernicke et al. | Treatment of eating disorders by |
| | | nerve stimulation |
| 6,540,789 | Silverman | Method of treating morbid obesity |
| 2003/0109935 Al | Geitz | Intragastric prosthesis for treatment |
| | | of morbid obesity |
| 2003/0109931 Al | Geitz | Intragastric stent for duodenum |
| | | bypass |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY OF THE INVENTION

In general, the invention is directed to medical devices and methods for biasing stretch receptors in the stomach wall of a patient to treat obesity. Biasing of the stretch receptors by pre-stretching induces an early sensation of satiety, causing the patient to consume less food. In accordance with the invention, biasing of the stretch receptors can be achieved by the implantation of bulking devices within the wall of the stomach. For example, the bulking devices may be placed in the mucosa, submucosa, or muscle layer of the stomach fundus or corpus. The bulking devices may be expandable and, in some embodiments, may take the form of a hydrogel material that expands following implantation in a muscle layer of the stomach.

Various embodiments of the present invention provide solutions to one or more problems existing in the prior art with respect to prior techniques for treatment of obesity. The problems include, for example, the limited efficacy and side effects of conventional appetite suppressant medications, and the need for potential repeated dosages of such medications by the patient. Additional problems relate to the general undesirability, invasiveness, infection risk, and recovery time associated with conventional surgical techniques for treatment of obesity, such as gastric reduction and bypass surgery, and other techniques for altering the shape or size of the stomach. Side effects of some invasive procedures, such as vomiting and “dumping syndrome,” are also undesirable. Further problems relate to the need for chronic implant of prostheses within the interior of the stomach to induce satiety, and the limited effectiveness of such prostheses.

Various embodiments of the present invention are capable of solving at least some of the foregoing problems. For example, a medical device and method in accordance with invention can provide a treatment for obesity that presents greater efficacy and lesser side effects, relative to administration of conventional appetite suppressant medications. In some embodiments, the invention may be capable of endoscopic deployment via the esophagus, and can thereby avoid the need for invasive surgical procedures. In this manner, the invention may also be capable of avoiding substantial reconstruction of the stomach, and offer reduced damage, recovery time, and side effects. Moreover, the invention does not require the presence of a chronically implanted prosthesis within the interior of the stomach.

Various embodiments of the invention may possess one or more features to solve the aforementioned problems in the existing art. In some embodiments, a method for treatment of obesity comprises implanting one or more bulking devices in the wall of the stomach of a patient, e.g., in the mucosa, submucosa or muscle layer. The implanted bulking devices are sized to stretch the muscle layer to an extent sufficient to bias stretch receptors and thereby induce a sensation of satiety in the patient. The bulking devices may be implanted by laparoscopic surgical techniques or endoscopically implanted via an esophagus of the patient.

The bulking devices may be expandable following implantation. In some embodiments, the bulking device includes a solid, hydrogel material that is expandable. In particular, the hydrogel material may be at least partially dehydrated prior to implantation, and then expand substantially due to rehydration following implantation. The hydrogel material may be constructed to produce a variety of shapes, sizes, and expansion ratios. A plurality of the bulking devices can be implanted at spaced apart positions within the wall of the stomach, e.g., in the fundus or corpus, to pre-stretch the stomach wall and thereby trigger stretch receptors to induce a sensation of satiety in the patient.

The invention may be embodied as a medical device for treatment of obesity, in which case the device may include an endoscopic delivery device sized for esophageal introduction into a stomach of a patient, and a bulking device for implantation in a stomach wall of the patient. A placement tool, deliverable via the endoscopic delivery device, implants the bulking device in the wall of the patient's stomach. As examples, the placement tool may take the form of a gripping member that grips the bulking device, or a needle through which the bulking device is delivered.

In comparison to known implementations of devices and method used for the treatment of obesity, various embodiments of the invention may provide one or more advantages. By pre-stretching the wall of the stomach, a medical device in accordance with the invention induces a sensation of satiety at an earlier point during the consumption of a meal by the patient.

Bulking devices implanted in the stomach wall trigger stretch receptors to bias the stomach into a predisposed state of apparent stretching, causing early onset of satiety. In this manner, the invention is capable of discouraging excessive consumption of food without the use of appetite suppressant medications, or chronic implantation of prostheses within the interior of the stomach.

Also, in some embodiments, implantation of the bulking devices can be achieved endoscopically without the need for invasive surgical intervention and substantial modification of the stomach structure. Consequently, the invention can reduce side effects, recovery time, and possibly eliminate hospital stays.

In various embodiments, the pre-stretched condition of the stomach wall can activate stretch receptors to provide, in effect, an early warning system for cessation of meal consumption. Consequently, the invention can counteract increased obesity and promote weight loss among obese patients.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a cross-sectional diagram of the interior of a stomach with implanted bulking devices in an expanded state to bias stretch receptors.

FIG. 2 is a diagram of the exterior of a stomach with implanted bulking devices.

FIG. 3 is a cross-sectional diagram of the interior of a stomach with implanted bulking devices in an unexpanded state.

FIG. 4 is a diagram illustrating deployment of an endoscopic delivery device shown in conjunction with a stomach of a patient.

FIG. 5 is a cross-sectional diagram of the interior of a stomach illustrating formation of an implantation pocket in the wall of the stomach.

FIG. 6 is a cross-sectional diagram of the interior of a stomach illustrating implantation of a bulking device in the wall of the stomach with a gripping device.

FIG. 7 is a cross-sectional diagram of the interior of a stomach illustrating implantation of a bulking device in the wall of the stomach with a needle.

FIG. 8 is a cross-sectional diagram of the interior of a stomach illustrating implantation of spherically shaped bulking devices in an unexpanded state.

FIG. 9 is a cross-sectional diagram of the interior of a stomach illustrating spherically shaped bulking devices in an expanded state following implantation.

FIG. 10 is a diagram of the exterior of a stomach with implanted bulking devices having irregular shapes with multiple lobes.

FIG. 11A is a plan view illustrating an irregularly shaped bulking device as shown in FIG. 10.

FIG. 11B is a cross-sectional side view of the irregularly shaped bulking device taken along line A-A′ of FIG. 11A.

FIG. 12 is a diagram illustrating a method for implanting a bulking device in a wall of a stomach to bias stretch receptors.

### DETAILED DESCRIPTION OF THE PREFERRED EMBODIMENTS

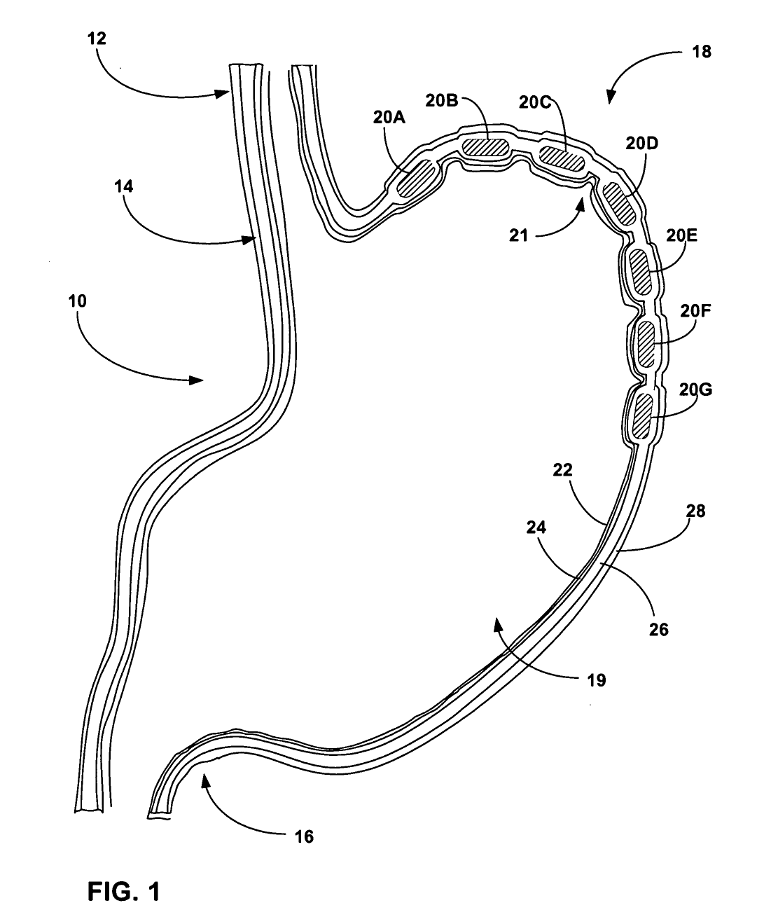

FIG. 1 is a cross-sectional diagram of the interior of a stomach 10, including esophagus 12, lower esophageal sphincter 14, pyloric sphincter 16, fundus 18, and corpus 19, with bulking devices 20A-20G (hereinafter bulking devices 20) implanted in stomach wall 21. For example, bulking devices 20A-20G can be implanted in the muscle layer of the stomach. Alternatively, bulking devices 20A-20G can be implanted in the mucosa or submucosa of stomach 10. In some embodiments, bulking devices 20 may be formed from an expandable material that is endoscopically or laparoscopically delivered and implanted in an unexpanded state. For example, bulking devices 20 may be formed from a hydrogel material that is implanted in an at least partially dehydrated state having a reduced size. Upon rehydration following implantation, bulking devices 20 assume an expanded state and increased size.

In FIG. 1, bulking devices 20 are depicted in an enlarged state with a size sufficient to bias stretch receptors within stomach wall 21. Each of bulking devices 20 exerts a localized stretching force on stretch receptors in stomach 10. The stretch receptors are coupled to the enteric nervous system of a patient. When triggered by the stretching force, the stretch receptors induce a sensation of satiety in the patient, and discourage the patient from consuming an excessively large meal. The role of stretch receptors in human gastric function is discussed, for example, in A. S. Paintal, “A study of gastric stretch receptors; their role in the peripheral mechanism of satiation of hunger and thirst,” J Physiol. Nov. 29, 1954; 29;126(2):255-70.

Bulking devices 20 bias stomach wall 21 into a pre-stretched condition that either triggers the stretch receptors or causes earlier triggering of the stretch receptors during the consumption of a meal. Hence, even though the stomach may not contain a substantial portion of food at the outset of a meal, implanted bulking devices 20 have already biased the stretch receptors into a condition that simulates the presence of a substantial portion of food. Consequently, during the course of a meal, stomach 10 requires a smaller amount of food to produce a sensation of satiety, which causes the patient to stop eating.

In this manner, bulking devices 20 do not significantly change the size or contents of stomach 10, but provide a physiological modification of stomach wall 21. This modification affects the response of the patient's enteric nervous system and the amount of food consumed by the patient, thereby preventing increased obesity and possibly causing or assisting in weight loss. In some cases, bulking devices 20 may be explanted after a desired course of obesity treatment has been achieved.

Bulking devices 20 may be endoscopically implanted, avoiding the need for surgery. As further shown in FIG. 1, bulking devices 20 may be implanted within stomach wall 21 throughout fundus 18 at spaced apart positions to provide localized stretching at several different points. Bulking devices 20 may be implanted in other regions of stomach 10, other than fundus 18, such as corpus 19. However, stretch receptors tend to be concentrated within fundus 18. Accordingly, in some embodiments, bulking devices 20 may be primarily or solely provided within fundus 18, where they are expected to be most effective in biasing stretch receptors. In other words, according to some embodiments, bulking devices 20 may be generally located only within fundus 18 and nowhere else. In other embodiments, bulking devices 20 may be implanted within fundus 18 and corpus 19, or solely within corpus 19.

Bulking devices 20 are implanted within stomach wall 21. Stomach wall 21 of a human stomach 10 generally includes four layers. With reference to FIG. 1, the innermost layer, mucosa 22, generates digestive juices. Submucosa 24 contains blood vessels that provide blood and oxygen to mucosa 22. Muscularis 26, a smooth muscle layer embedded with nervous plexus, contracts to mix food with digestive juices generated by mucosa 22. Serosa 28, the fourth and outermost layer, protects the other layers and confines digestive juices to stomach 10.

As one example, bulking devices 20 may be implanted within muscularis 26, which contains the stretch receptors. The stretch receptors are coupled to the nervous system via the vagus nerves, and signal the patient when stomach 10 reaches a stretch point indicating a large quantity of food. With bulking devices 20, the patient perceives that the stomach has reached a stretch point indicating fullness much earlier during the course of the meal and at a point at which the stomach is not actually full. In other embodiments, bulking devices 20 may be implanted within mucosa 22 or submucosa 24.

Bulking devices 20 may be implanted surgically from the serosal aspect of stomach 10 (i.e., from the outer surface) or endoscopically from the mucosal aspect of the stomach (i.e., from the inside surface) of the stomach. Surgical implantation may involve laparoscopic techniques. As discussed above, however, it may be highly desirable to implant bulking devices 20 via the esophagus 12 using an endoscopic delivery device. In this manner, a highly or even minimally invasive surgery can be avoided, and recovery time can be shortened. Rather, the esophagus 12 of the patient may be intubated with the endoscopic delivery device via the oral or nasal passage under general anesthesia.

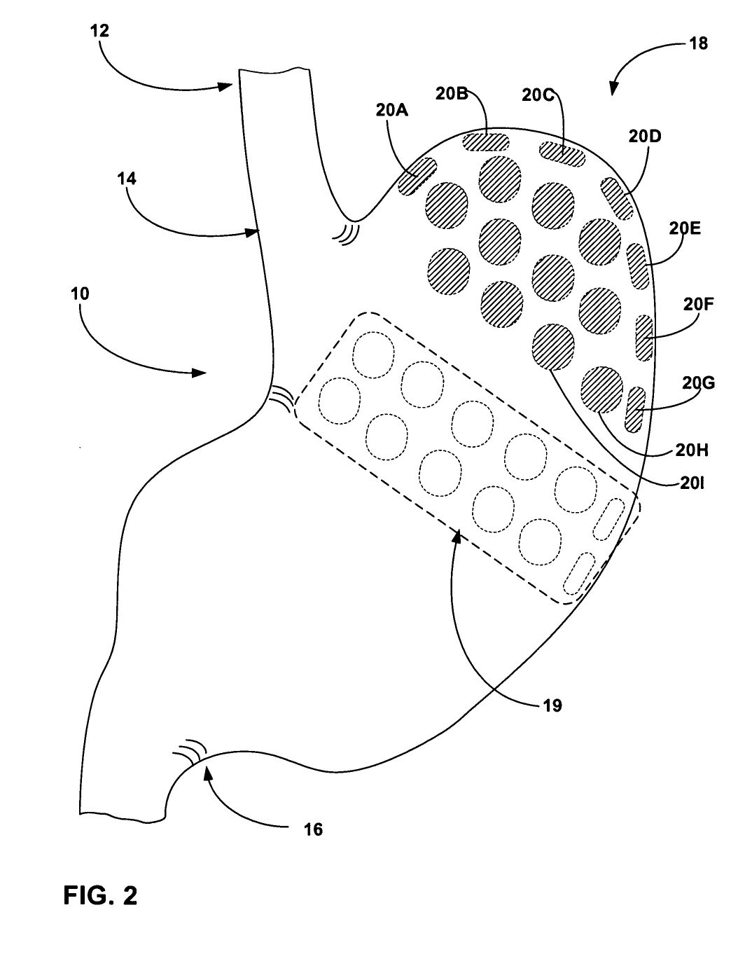

FIG. 2 is a diagram of the exterior of stomach 10 with implanted bulking devices 20. As shown in FIG. 2, bulking devices 20 may be implanted at spaced apart positions within fundus 18 of stomach 10. FIG. 2 includes additional reference numerals 20H, 20I to identify some of the additional bulking devices 20 in the wall 21 of stomach 10. Bulking devices 20 may be implanted on an anterior, posterior and lateral wall of fundus 18 so as to extend generally about the entire fundus region. Also, in some embodiments, bulking devices 20 may be implanted in the corpus, as indicated by the bulking devices associated with reference numeral 19 in FIG. 2.

FIG. 2 depicts only an anterior side of fundus 18 for ease of illustration. It should be understood, however, that an array of bulking devices 20 as depicted on the anterior side may likewise be implanted on a posterior side of fundus 18, or corpus 19. In other embodiments, bulking devices 20 may be implanted on a single side or two sides, i.e., posterior, anterior, and/or lateral. In each case, bulking devices 20 are implanted as relatively small solid objects that then expand when they rehydrate following implantation, and thereby bias the stretch receptors in fundus 18 of stomach 10.

In the example of FIG. 2, bulking devices 20 have a substantially disc-like shape. In other embodiments, bulking devices 20 may have a variety of shapes, e.g., substantially spherically shaped, rod- or cylinder-shaped, or irregularly shaped, as will be described. In an at least partially dehydrated state for implantation, the disc-like shape of bulking devices 20 in FIG. 2 may have a thickness of approximately 1 mm to 2 mm, and a diameter of approximately 10 mm to 15 mm. Following implantation in stomach wall 21 and subsequent rehydration, the disc-like shape may expand to have a thickness of approximately 4 mm to 6 mm, and a diameter of approximately 8 mm to 10 mm. Hence, in some embodiments, the disc-like shape of bulking device 20 may exhibit expansion in thickness, but contraction in diameter, following implantation.

In an at least partially dehydrated state for implantation, disk-like bulking devices 20 may have a volume in a range of approximately 75 mm³ to 350 mm³. Upon expansion following implantation, bulking devices 20 may have a volume in a range of approximately 200 mm³ to 470 mm³. Hence, each bulking device 20 may have a volumetric expansion ratio, from an at least partially dehydrated state (pre-implantation) to a hydrated, expanded state (post-implantation), of at least approximately 4.5:1, and more particularly approximately 27:1.

As a further illustration, if constructed as an elongated rod- or cylinder-like member, the hydrogel material may exhibit pre-implantation dimensions of less than or equal to approximately 2 mm in diameter by approximately 20 mm in length, and post-implantation dimensions of greater than or equal to approximately 6 mm in diameter by approximately 15 mm. This corresponds to an exemplary pre-implantation volume of less than approximately 65 mm³, and a post-implantation volume of greater than or equal to approximately 400 mm³.

Hence, in general, the pre-implantation volume of a bulking device 20 is less than or equal to 100 mm³ and the post-implantation volume of a bulking device is greater than 200 mm³. In some embodiments, the pre-implantation volume of bulking device 20 is less than or equal to approximately 75 mm³, and the post-implantation volume of the bulking device is greater than or equal to approximately 300 mm³.

Spacing between adjacent bulking devices 20 may be controlled by taking into account the expanded size of the bulking devices. The outer perimeters of adjacent, expanded bulking devices 20 may be separated by a distance in range of approximately 3 mm to 10 mm, and more particularly approximately 3 mm to 5 mm. Adjacent bulking devices 20 are separated by a section of intact muscularis, mucosa or submucosa, and provide a localized stretching effect. By leaving a substantial portion of the muscularis, mucosa, or submucosa intact, bulking devices 20 can bias the stretch receptors without compromising the contractile function of the stomach wall 21 in support of the digestion process.

In other embodiments, however, an array of bulking devices 20 may be placed so that, upon expansion, the outer perimeters of the bulking devices actually come into contact with one another. In this manner, bulking devices 20 may cooperate to provide an overall stretching effect to a larger region of fundus 18. Bulking devices 20 may be placed in a plurality of regions, while leaving other areas of the muscularis between regions intact.

Whether bulking devices are spaced apart or implanted to contact one another upon expansion, the bulking devices do not expand wall 21 of stomach 10 like consumption of a meal would, in which case the entire stomach wall would tend to stretch outward as a unitary body. Instead, bulking devices 20 provide localized or regional stretching of selected portions of fundus 18 to trigger the stretch receptors, and cause a false sensation of fullness that induces early satiety.

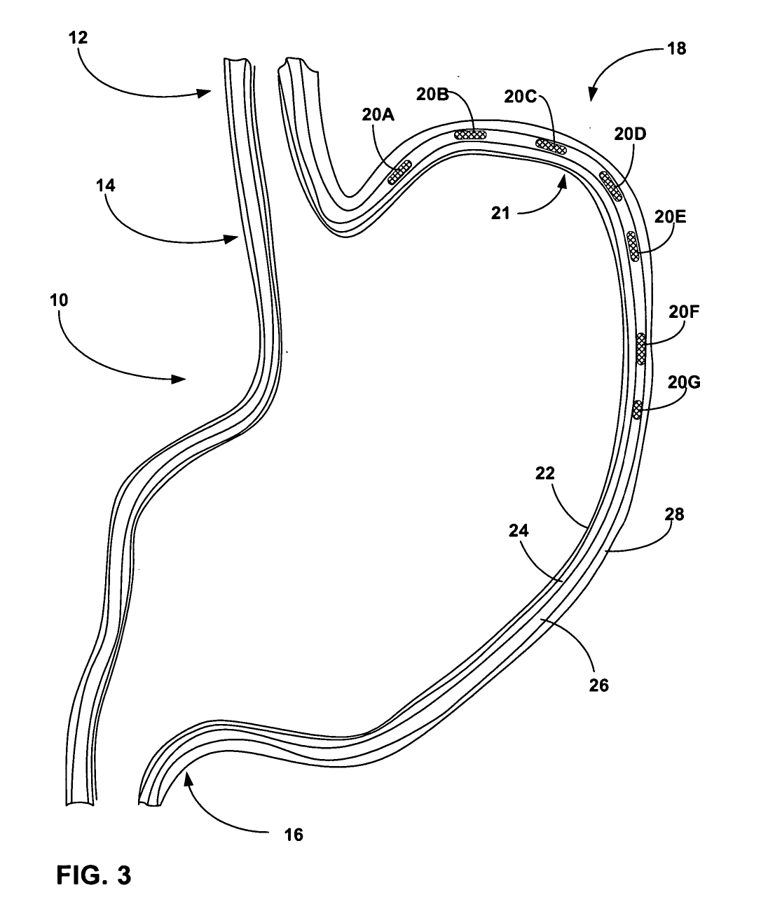

FIG. 3 is a cross-sectional diagram of the interior of a stomach with implanted bulking devices 20 in an unexpanded state, e.g., an at least partially dehydrated state in the event bulking devices 20 are formed from a hydrogel material. Bulking devices 20 correspond to the bulking devices of FIGS. 1 and 2, but represent the reduced size of the bulking devices as they are implanted in stomach wall 21. In particular, as mentioned above, bulking devices 20 may be implanted as solid, hydrogel materials in an at least partially dehydrated state. Accordingly, as shown in FIG. 3, the size of bulking devices 20 upon implantation is much less than the size of the bulking devices following implantation, in terms of volume. The expansion of bulking devices 20 occurs as the at least partially dehydrated hydrogel material takes on moisture from within stomach wall 21 and rehydrates to assume an enlarged size.

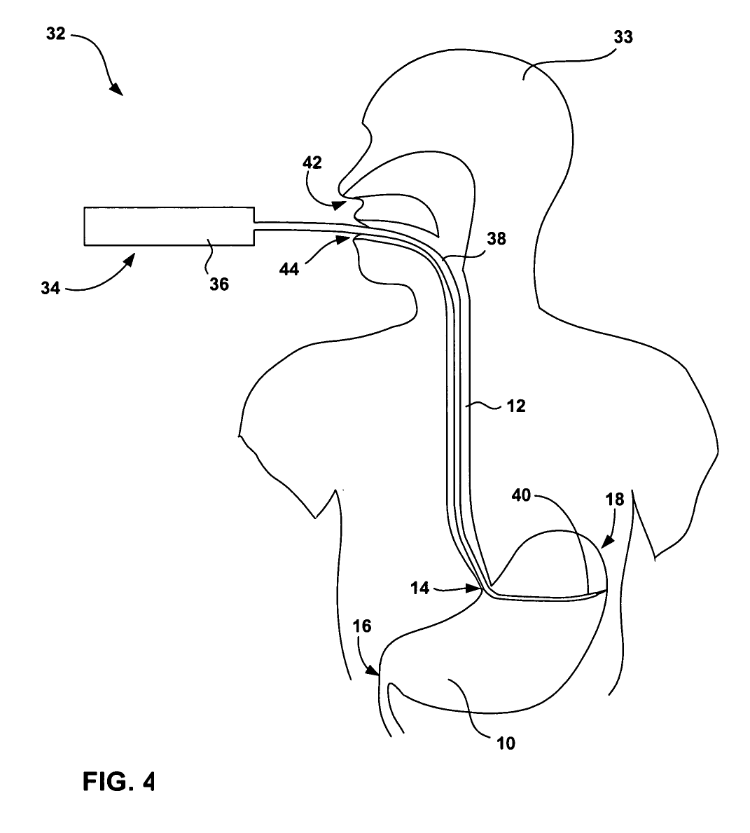

FIG. 4 is a diagram illustrating deployment of an endoscopic delivery system 32 shown in conjunction with a stomach 10 of a patient 33. As shown in FIG. 4, esophageal delivery system 32 serves to position and place bulking devices 20 (FIGS. 1-3) within the stomach 10 of patient 33. Esophageal delivery device 32 includes an endoscopic delivery device 34 having a proximal portion, referred to herein as a handle 36, and a flexible probe 38 that extends from handle 36 into the gastrointestinal tract of patient 33. A bulking device 20 is delivered to a target location in stomach wall 21 of fundus 18 via a distal end 40 of flexible probe 38. Precise positioning may be aided by endoscopic viewing provided by an imaging endoscope integrated within or delivered simultaneously with flexible probe 38. In addition, external imaging techniques such as fluoroscopy or ultrasonic imaging may be used to aid precise positioning. Distal end 40 of delivery device 34 enters esophagus 12, via either nasal cavity 42 or oral cavity 44, and extends into stomach 10 to a desired placement location.

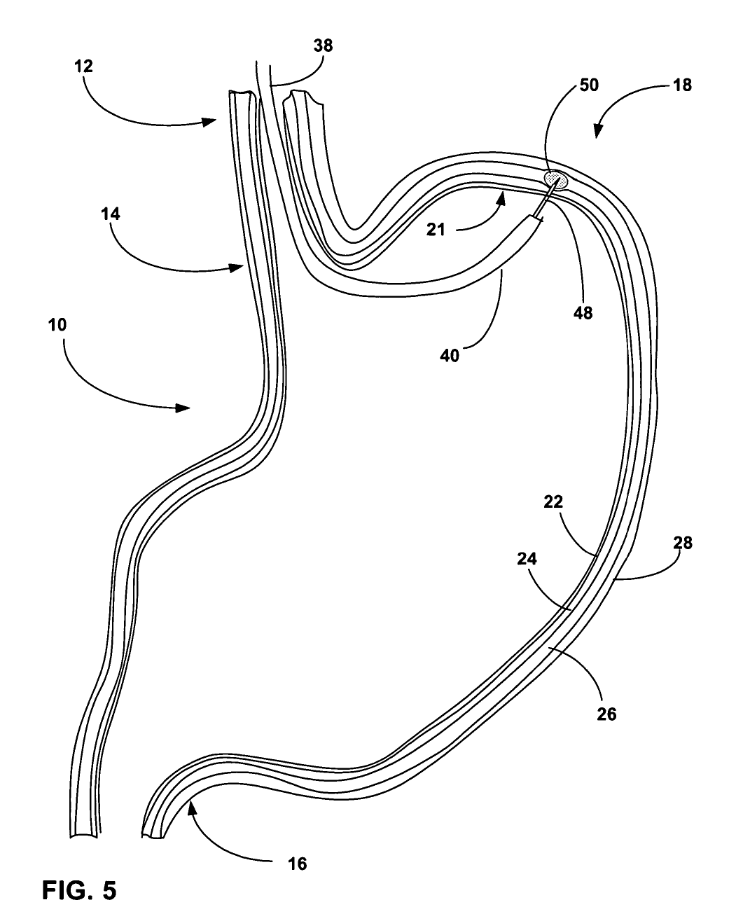

FIG. 5 is a cross-sectional diagram of the interior of a stomach illustrating the optional formation of an implantation pocket in wall 21 of stomach 10. Formation of an implantation pocket prior to implantation of bulking device 20 may not be necessary. As will be described, however, the formation of an implantation pocket may be advantageous in that it serves to stretch an area in the muscularis 26 to provide space to receive the bulking device 20, and perhaps accommodate some of the expansion of the bulking device.

As shown in FIG. 5, a physician extends a needle along the length of flexible probe 38 and out distal end 40. The physician may steer distal end 40 of flexible probe 28 to a desired location on stomach wall 21 using conventional endoscopic steering equipment, such as embedded pre-formed wires or the like. Upon penetration of stomach wall 21, e.g., to a depth coincident with muscularis 26, the physician injects a bolus 50 of saline or other biocompatible fluid to expand a localized region of the muscularis and create the implantation pocket. Other depths may be appropriate for mucosal or submucosal implantation.

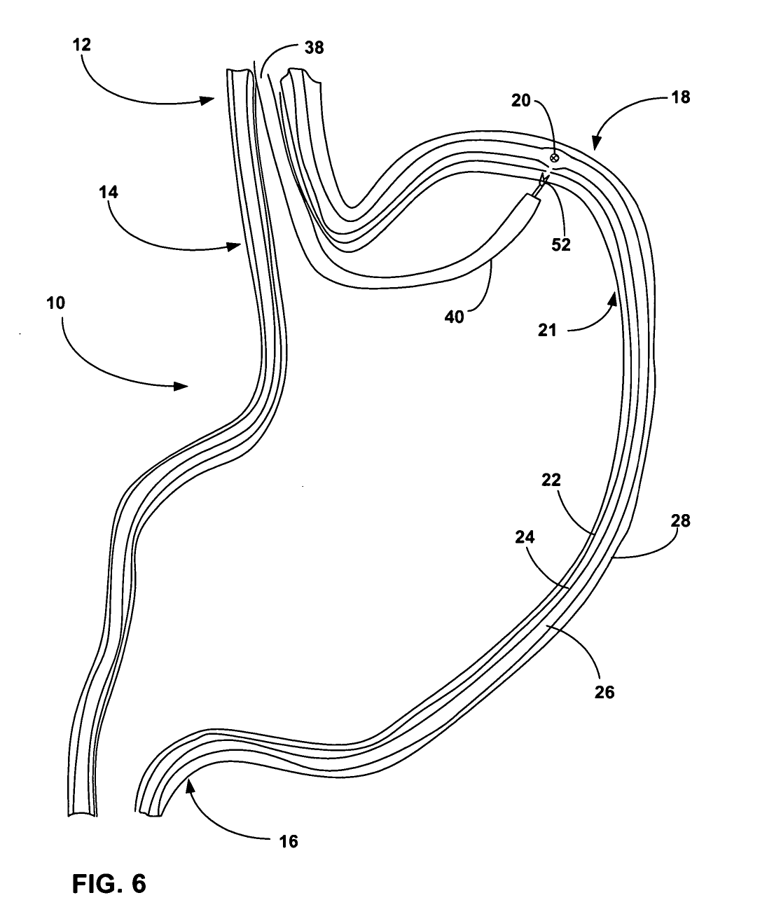

FIG. 6 is a cross-sectional diagram of the interior of a stomach 10 illustrating implantation of a bulking device 20 in wall 21 of the stomach with a gripping device 52. Following formation of an implantation pocket, as shown in FIG. 5, the physician inserts an elongated placement tool into flexible probe 38. A distal end of the placement tool may include a gripping device 52, e.g., a device having a pair of jaws or forceps to grip and place a bulking device 20. As shown in FIG. 6, the physician places bulking device 20, in its at least partially dehydrated state, into stomach wall 21, and then withdraws gripping device 52. In some embodiments, the physician may deploy a suturing device via flexible probe 38 to close the implantation hole formed in stomach wall 21.

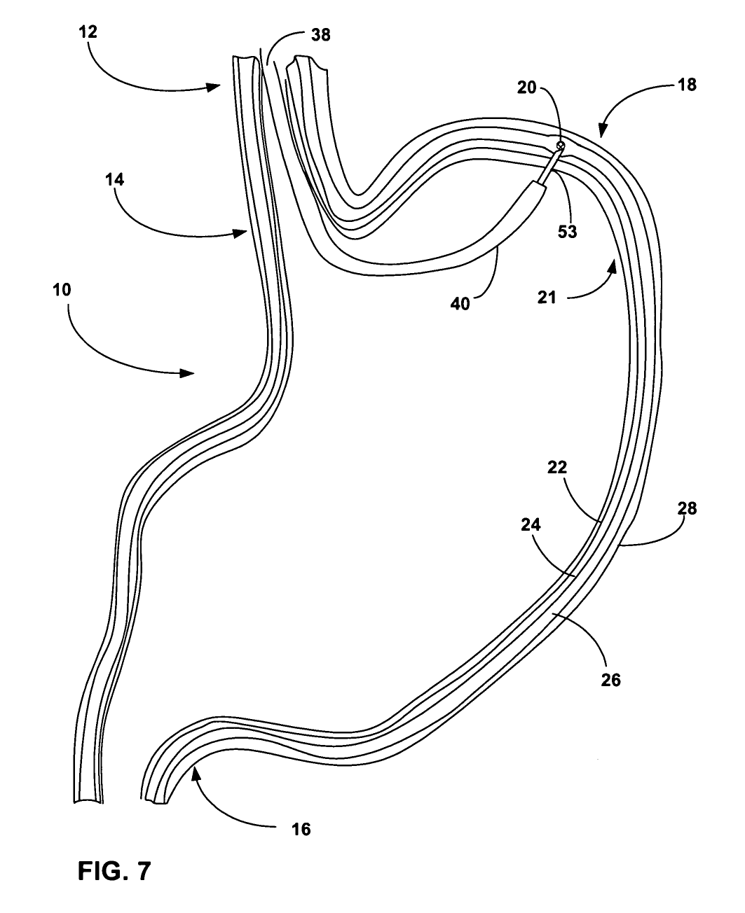

FIG. 7 is a cross-sectional diagram of the interior of a stomach illustrating implantation of a bulking device 20 in the wall 21 of the stomach 10 with a needle 53. FIG. 7 generally conforms to FIG. 6, but depicts the use of a needle 53 to implant bulking device 20 rather than a gripping device 52 (FIG. 6). In this example, bulking device 20 is initially sized small enough to fit within the bore of a needle 53. For example, bulking device 20 may be a spherical or rod-shaped element formed from an at least partially dehydrated hydrogel material.

Bulking device 20 may be initially mounted in a tip of needle 53 prior to introduction of the needle into stomach 10 via flexible probe 38. Upon placement of the tip of needle 53 with stomach wall 21, the physician expels bulking device 20 from the needle. The physician may actuate a fluid pressure source or elongated push rod to drive bulking device 20 out of needle 53 and into muscularis 26. Following implantation via needle 53, bulking device 20 expands, e.g., by rehydration, to assume an enlarged size sufficient to bias stretch receptors within stomach wall 21. Then, needle 53 and flexible probe 38 may be withdrawn or repositioned to implant another bulking device 20 at a different tissue site within the stomach wall 21.

Needle 53 may be withdrawn from flexible probe to reload the tip of the needle with a bulking device 20. Alternatively, needle 53 may be initially loaded with several bulking device 20 in a stack within the needle lumen. In this case, the physician advances a push rod by a finite distance or applies fluid pressure in a metered amount to expel bulking devices 20 one at a time as needle 53 is repositioned. In this manner, the physician may place a plurality of bulking devices 20 within stomach wall 21 without withdrawing flexible probe 38 and needle 53.

As an example, needle 53 may have a diameter in the range of less than approximately 2 mm to 4 mm in inside diameter, which can accommodate a spherical or rod-like bulking device 20 having a diameter or transverse cross-section, respectively, of approximately 1.5 mm to 3.5 mm in diameter. Upon implantation of bulking device 20 with needle 53, the implantation hole may be sufficiently small that there is not a need for suturing or stapling. Instead, needle 53 may proceed among a plurality of implantation sites and then be withdrawn with flexible probe 38 of endoscopic delivery device 34. As an alternative, however, needle 53 may be electrically conductive and coupled to a source of electrical current to apply cautery energy (e.g., in conjunction with an external electrode pad) to each implantation site as the needle is withdrawn.

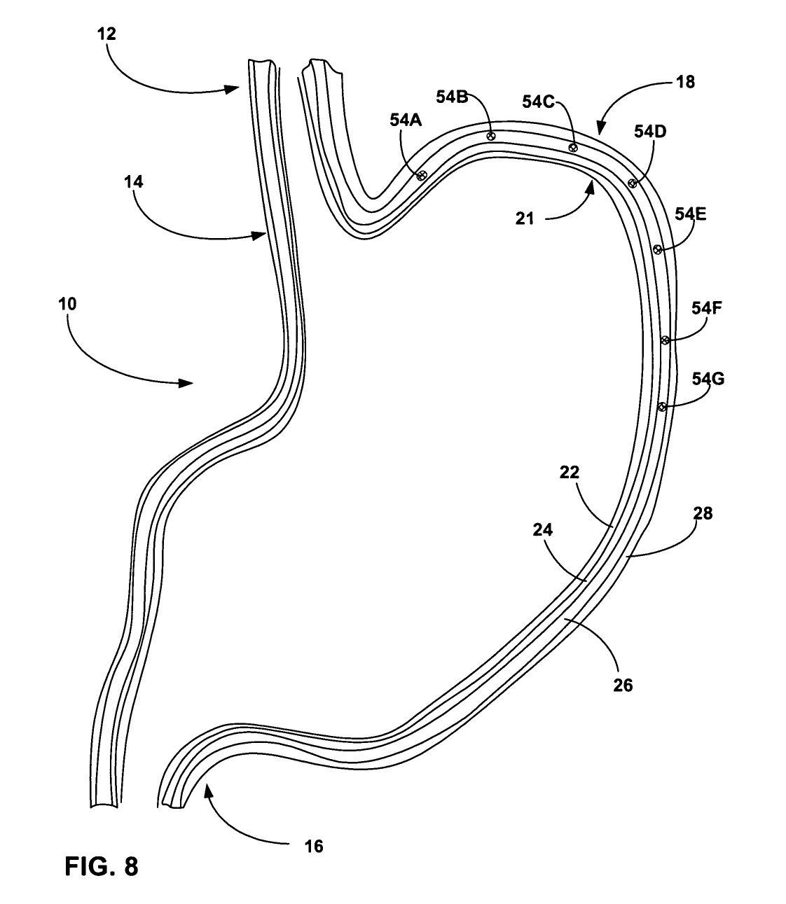

FIG. 8 is a cross-sectional diagram of the interior of a stomach 10 illustrating implantation of spherically shaped bulking devices 54A-54G (herein spherical bulking devices 54). As shown in FIG. 8, spherical bulking devices 54 are initially implanted in a contracted state. For example, if fabricated from a hydrogel material, spherical bulking devices may be implanted initially in an at least partially dehydrated state. Spherical bulking devices 54 may be implanted by a variety of methods, including laparoscopic surgical methods and the particular endoscopic methods described herein.

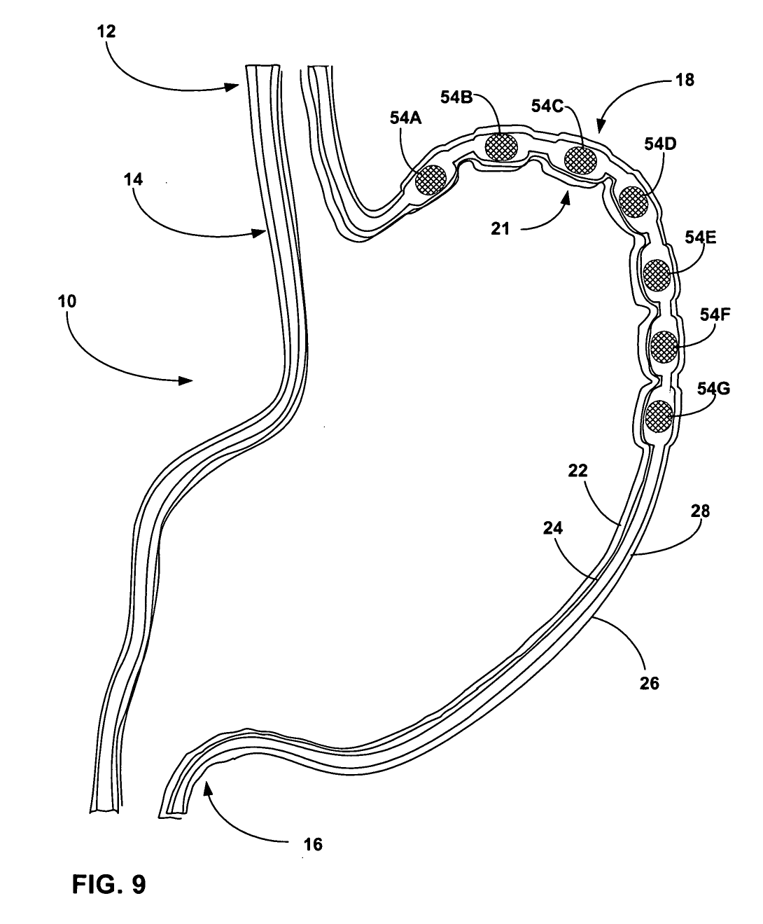

FIG. 9 is a cross-sectional diagram of the interior of a stomach 10 illustrating spherical bulking devices 54 in an expanded state following implantation. If spherical bulking devices 54 are constructed from a hydrogel material, for example, the bulking devices expand upon rehydration following implantation within stomach wall 21. In an at least partially dehydrated state for implantation, the substantially spherical shape of bulking device 54 may have a diameter of approximately 1 mm to 4 mm and more particularly approximately 1 mm to 2.5 mm. Following implantation in stomach wall 21, the spherical bulking device 54 may have a diameter of approximately 4 mm to 16 mm, and more particularly a diameter of approximately 4 mm to 10 mm.

In an at least partially dehydrated state for implantation, spherical bulking devices 54 may have a volume in a range of approximately 0.5 mm³ to 33 mm³, and more particularly approximately 0.5 mm³ to 8.2 mm³. Upon expansion following implantation, bulking devices 54 may have a volume in a range of approximately 33 mm³ to 2143 mm³ and more particularly approximately 33 mm³ to 523 mm³. Hence, each spherical bulking device 54 may have an expansion ratio, from an at least partially dehydrated state (pre-implantation) to a hydrated, expanded state (post-implantation), of at least approximately 1:4, and possibly much higher.

As in the embodiments of FIGS. 1-3, bulking devices 54 may be implanted such that a space exists between adjacent bulking devices, or implanted more closely so that at least some of the bulking devices contact one another. Disc-shaped bulking devices 20, as shown in FIGS. 1-3, provide a more gradual stretching profile within muscularis 26, due to the gradual transition in thickness across the radius of the disc-like shape. In this manner, tissue containing stretch receptors in the vicinity of disc-shaped bulking device 20 are stretched to a similar amount. On the contrary, spherical bulking devices 20 provide a more acute stretching profile, and stretch tissue in the vicinity of the spherical shape to a varying degree.

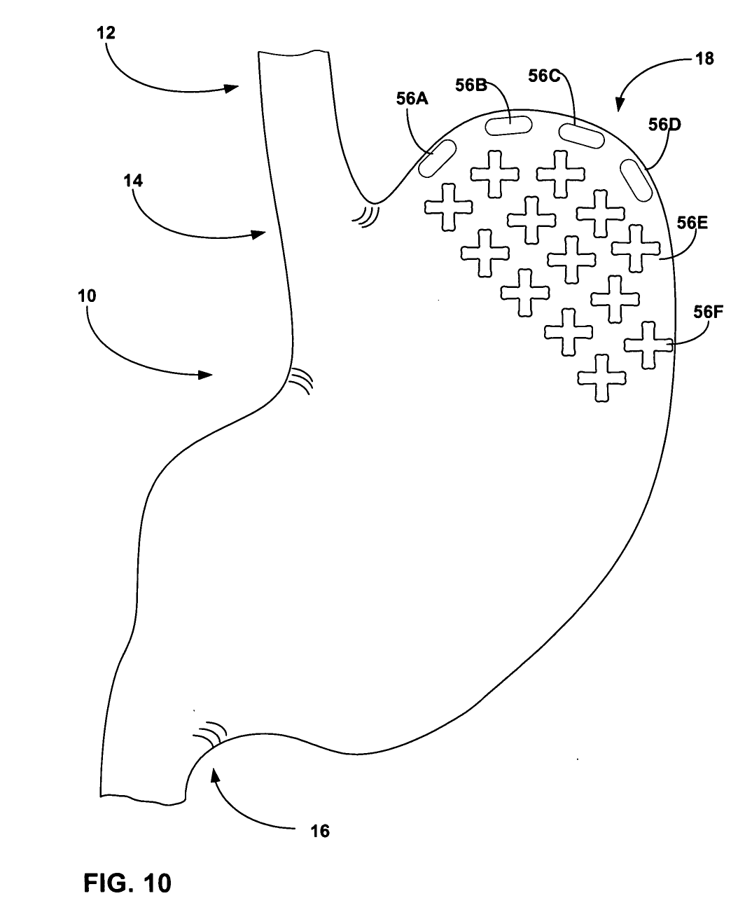

FIG. 10 is a diagram of the exterior of a stomach with implanted bulking devices 56A-56F (hereinafter bulking devices 56) having irregular shapes with multiple lobes. Bulking devices 56 may be subject to a variety of different irregular shapes that deviate from a regular shape, such as the disc, spherical, or rod-like shapes described herein. In the example of FIG. 10, each of the bulking devices has a substantially cross-like shape with four separate lobes that extend outward from a central point.

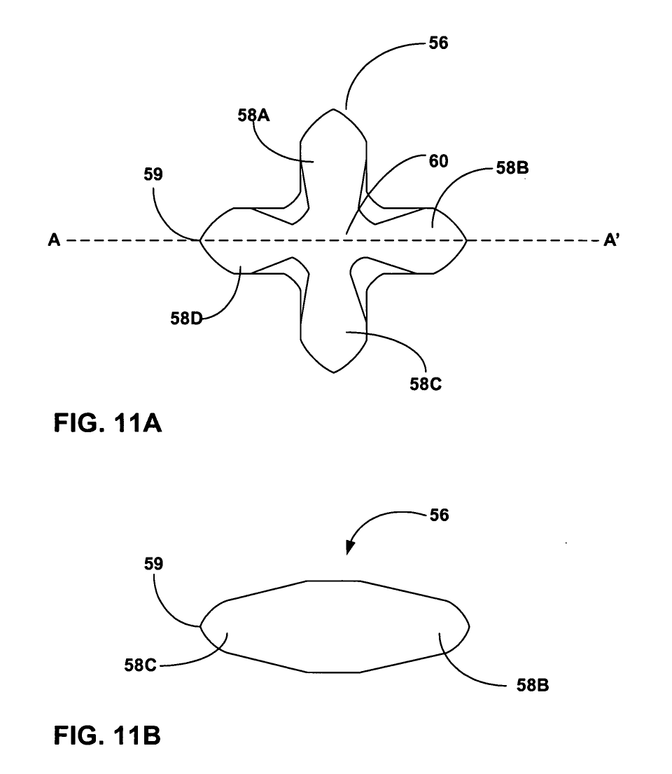

FIG. 11A is a plan view illustrating an irregularly shaped bulking device 56 as shown in FIG. 10. FIG. 11B is a cross-sectional side view of the irregularly shaped bulking device 56 taken along line A-A′ of FIG. 11A. As shown in FIGS. 11A and 11B, bulking devices includes lobes 58A, 58B, 58C, 58D. Each lobe may taper upward from a narrower thickness at an outer perimeter 59 to a larger thickness at a central region 60. In some embodiments, an irregularly shape bulking device 56, such as the cross-like shape, may permit more dense or ordered packing of adjacent bulking devices.

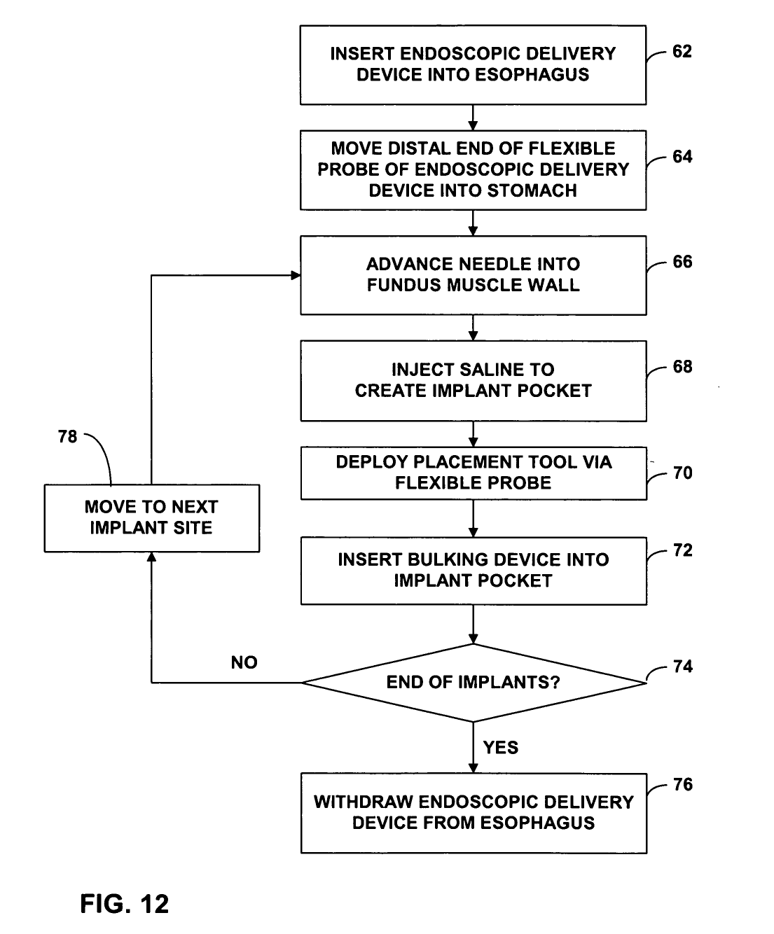

FIG. 12 is a diagram illustrating a method for implanting a bulking device in a wall 21 of a stomach 10 to bias stretch receptors. A physician inserts an endoscopic delivery device into the esophagus of a patient (62), and moves a distal end of a flexible probe into the stomach of the patient (64). The physician then advances a needle from the distal end of the flexible probe and into a musoca, submucosa or muscle layer in the fundus of the stomach (66). Once the distal tip of the needle is in place, the physician injects saline into the stomach wall to create an implantation pocket (68). As discussed above, bulking devices may also, or alternatively, be implanted in the corpus of the stomach. Accordingly, the method described with respect to FIG. 12 may likewise be practiced in the corpus.

The physician then withdraws the needle, and deploys a placement tool via the flexible probe (70), and implants the bulking device into the implant pocket (72). The placement tool may take the form of a needle or gripping device. If additional bulking devices are to be implanted (74), the physician repositions the flexible probe to another implant site (78) and repeats the implantation process. When all bulking devices have been implanted, the physician withdraws the endoscopic delivery device from the esophagus (76).

A bulking device, as described herein, preferably is soft and compliant to minimized trauma within stomach wall 21 upon implantation. The bulking device may be constructed from a variety of biocompatible polymeric materials. Again, the materials forming bulking device may be expandable. In particular, as described herein, the bulking devices may be formed from an expandable hydrogel material. Suitable materials, including hydrogel materials, are described in U.S. Pat. No. 6,401,718 to Johnson et al., assigned to Medtronic Endonetics, Inc., and entitled “Submucosal esophageal bulking device,” the entire content of which is incorporated herein by reference. Suitable techniques and components for implantation of bulking devices are also described in Johnson et al., and may be adapted for use in implantation of bulking devices in stomach 10 in accordance with the present invention.

As alternatives, described in Johnson et al., bulking device 20 may take the form of a fluid-filled, flexible capsule, pillow or balloon made from elastomeric materials such as silicone, latex, urethane, and the like. Example fillers include biocompatible liquid or gel such as saline, silicone oil, DMSO, polyvinyl, pyrollidone and hydrogels. As a further alternative, the bulking device may be a unitary structure formed by molding, casting, stamping or the like. The unitary structure may formed from hydrogel material, biocompatible foam material such as silicone foam or polyurethane foam, or a variety of biocompatible materials such as silicone, polyurethane, polysulfone, polyester, and the like. As described in Johnson et al., foam material may include outer skin of porous foam that facilitates tissue ingrowth.

As alternatives to implanted solid materials, bulking devices may be formed by injected fluids or gels that form solids or semi-solids following injection. A variety of implanted solid materials and injected fluids suitable for formation of bulking devices to bias stretch receptors, as described herein, are disclosed in U.S. Published patent application No. 20040019388, to Starkebaum, assigned to Medtronic, Inc. and entitled “Methods and implants for retarding stomach emptying to treat eating disorders,” the entire content of which is incorporated herein by reference. Accordingly, bulking devices may refer to solid, semi-solid, or filled implants, or injected fluids that formed solid or semi-solid bulking devices within wall 21 of stomach 10 to bias stretch receptors and thereby treat obesity.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems as described herein.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method for treatment of obesity, the method comprising implanting one or more bulking devices in a wall of a stomach of a patient, wherein the implanted bulking devices are sized to stretch the stomach wall to an extent sufficient to bias stretch receptors and thereby induce a sensation of satiety in the patient.

2. The method of claim 1, further comprising endoscopically implanting the bulking devices via an esophagus of the patient.

3. The method of claim 1, further comprising repositioning an endoscopic device among a plurality of implantation sites to endoscopically implant a plurality of the bulking devices.

4. The method of claim 1, wherein each of the bulking devices includes a solid, hydrogel material.

5. The method of claim 4, further comprising implanting the bulking devices with the hydrogel material in an at least partially dehydrated state, the bulking devices rehydrating upon implantation and thereby expanding in size to stretch the stomach wall.

6. The method of claim 1, wherein the bulking devices are expandable following implantation.

7. The method of claim 6, wherein the bulking devices are expandable from a pre-implantation volume of less than approximately 100 mm³ to a post-implantation volume of greater than or equal to approximately 200 mm³.

8. The method of claim 6, wherein the bulking devices are expandable from a pre-implantation size to a post-implantation size of greater than or equal to approximately 2 times the pre-implantation size.

9. The method of claim 1, wherein each of the bulking devices, following implantation, has a volume in a range of approximately 200 mm³ to approximately 470 mm³.

10. The method of claim 1, wherein each of the bulking devices is substantially spherical in shape, substantially disc-like in shape, or substantially rod-like in shape.

11. The method of claim 1, wherein each of the bulking devices includes three or more lobes.

12. The method of claim 1, further comprising implanting the bulking devices in a fundus of the stomach.

13. The method of claim 1, further comprising implanting the bulking devices in a mucosa, submucosa or muscle layer of the stomach wall.

14. The method of claim 1, further comprising implanting a plurality of the bulking devices in a fundus and a corpus of the stomach.

15. The method of claim 1, further comprising implanting a plurality of the bulking devices at spaced apart positions within the stomach wall.

16. The method of claim 1, further comprising injecting a fluid into the stomach wall to create an implantation pocket, and implanting one of the bulking devices in the implantation pocket.

17. The method of claim 1, further comprising implanting the bulking devices only in the fundus region.

18. The method of claim 1, wherein implanting the bulking devices includes injecting a fluid material into the stomach wall to form the bulking devices.

19. A system for treatment of obesity, the system comprising:
an endoscopic delivery device sized for esophageal introduction into a stomach of a patient;
a bulking device for implantation in a wall of the stomach, wherein the implanted bulking device is sized to stretch the stomach wall to an extent sufficient to bias stretch receptors and thereby induce a sensation of satiety in the patient; and
a placement tool, deliverable via the endoscopic delivery device, to implant the bulking device in the stomach wall.

20. The system of claim 19, wherein the bulking device includes a solid, hydrogel material.

21. The system of claim 19, wherein the solid, hydrogel material is in an at least partially dehydrated state prior to implantation, and the bulking devices rehydrates upon implantation and thereby expands in size to stretch the stomach wall.

22. The system of claim 19, wherein the bulking device is expandable from a pre-implantation volume of less than approximately 100 mm³ to a post-implantation volume of greater than or equal to approximately 200 mm³.

23. The system of claim 19, wherein the bulking device is expandable from a pre-implantation size to a post-implantation size of greater than or equal to approximately 2 times the pre-implantation size.

24. The system of claim 19, wherein the bulking device, following implantation, has a volume in a range of approximately 200 mm³ to approximately 470 mm³.

25. The system of claim 19, wherein the bulking device is substantially spherical in shape, substantially disc-like in shape, or substantially rod-like in shape.

26. The system of claim 19, wherein the bulking device includes three or more lobes.

27. The system of claim 19, wherein the endoscopic delivery device is steerable to orient the placement tool for implantation of the bulking devices in a fundus of the stomach.

28. The system of claim 19, wherein the placement tool implants the bulking device in a mucosa, submucosa or muscle layer of the stomach wall.

29. The system of claim 19, wherein the endoscopic delivery device is steerable to orient the placement tool for implantation of the bulking device in a fundus and a corpus of the stomach.

30. The system of claim 19, further comprising a needle, deliverable via the endoscopic delivery device, to inject a fluid into the stomach wall to create an implantation pocket for the bulking device.

31. The system of claim 19, wherein the bulking device comprises a fluid material for injection into the stomach wall to form the bulking device.

32. A system for treatment of obesity, the system comprising:
an endoscopic delivery device sized for esophageal introduction into a stomach of a patient;
means, implantable in a wall of the stomach, for stretching the stomach wall to an extent sufficient to bias stretch receptors and thereby induce a sensation of satiety in the patient; and
means for implanting the bulking device in the stomach wall of the patient via the endoscopic delivery device.

33. The system of claim 32, wherein the stretching means includes a solid, hydrogel material.

34. The system of claim 33, wherein the hydrogel material is in an at least partially dehydrated state prior to implantation, and the hydrogel material rehydrates upon implantation and thereby expands in size to stretch the stomach wall.

35. The system of claim 32, wherein the stretching means is expandable from a pre-implantation volume of less than approximately 100 mm³ to a post-implantation volume of greater than or equal to approximately 200 mm³.

36. The system of claim 32, wherein the stretching means is expandable from a pre-implantation size to a post-implantation size of greater than or equal to approximately 2 times the pre-implantation size.

37. The system of claim 32, wherein the stretching means, following implantation, has a volume in a range of approximately 200 mm³ to approximately 470 mm³.

38. The system of claim 32, wherein the stretching means is substantially spherical in shape, substantially disc-like in shape, or substantially rod-like in shape.

39. The system of claim 32, wherein the stretching means includes three or more lobes.

40. The system of claim 32, further comprising a needle, deliverable via the endoscopic delivery device, to inject a fluid into the stomach wall to create an implantation pocket for the stretching means.

41. The system of claim 32, wherein the endoscopic delivery device is steerable to orient the placement tool for implantation of the stretching means in a fundus of the stomach.

42. The system of claim 32, wherein the implanting means implants the bulking device in a mucosa, submucosa or muscle layer of the stomach wall.

43. The system of claim 32, wherein the endoscopic delivery device is steerable to orient the placement tool for implantation of the stretching means in a fundus and a corpus of the stomach.

44. The system of claim 32, wherein the stretching means includes a fluid material for injection into the stomach wall to form the stretching means.

45. A system for treatment of obesity, the system comprising:
an endoscopic delivery device sized for esophageal introduction into a stomach of a patient;
a solid, expandable hydrogel prosthesis, implantable in a wall of the stomach, to stretch the stomach wall to an extent sufficient to bias stretch receptors and thereby induce a sensation of satiety in the patient; and
an implant tool, deliverable via the endoscopic delivery device, to implant the bulking device in the stomach wall of the patient via the endoscopic delivery device.

46. The system of claim 45, wherein the hydrogel prosthesis is in an at least partially dehydrated state prior to implantation, and the hydrogel prosthesis rehydrates upon implantation and thereby expands in size to stretch the stomach wall.

47. The system of claim 45, wherein the hydrogel prosthesis is expandable from a pre-implantation volume of less than approximately 100 mm³ to a post-implantation volume of greater than or equal to approximately 200 mm³.

48. The system of claim 45, wherein the hydrogel prosthesis is expandable from a pre-implantation size to a post-implantation size of greater than or equal to approximately 2 times the pre-implantation size.

49. The system of claim 45, wherein the hydrogel prosthesis, following implantation, has a volume in a range of approximately 200 mm³ to approximately 470 mm³.

50. The system of claim 45, wherein the hydrogel prosthesis is substantially spherical in shape, substantially disc-like in shape, or substantially rod-like in shape.

51. The system of claim 45, further comprising a needle, deliverable via the endoscopic delivery device, to inject a fluid into the stomach wall to create an implantation pocket for the hydrogel prosthesis.
